(19) United States
(12) Patent Application Publication
(10) Pub. No.: US 2009/0191164 A1
(43) Pub. Date: Jul. 30, 2009

(54) HUMAN HEMATOPOIETIC MULTIPOTENT PROGENITOR CELLS

(76) Inventors: Ravindra Majeti, Standford, CA (US); Irving L. Weissman, Standford, CA (US)

Correspondence Address: Stanford University Office of Technology Licensing;Bozicevic, Field & Francis LLP, 1900 University Avenue, Suite 200, East Palo Alto, CA, 94303, US

(21) Appl. No.: 12/353,098

(22) Filed: Jan. 13, 2009

Related U.S. Application Data
(60) Provisional application No. 61/057,772, filed on May 30, 2008.
(60) Provisional application No. 61/011,324, filed on Jan. 15, 2008.

(51) Int. Cl. A61K 35/12; C12N 5/08; C12Q 1/68; C12Q 1/02; A61P 37/00

(52) U.S. Cl. 424/93.7; 435/366; 435/372; 435/6; 435/29

(57) ABSTRACT

A substantially enriched human multipotent progenitor cell population is provided, which is characterized as a progenitor cell capable of giving rise to the multipotent lineage but which lacks certain long-term self-renewal properties of the hematopoietic stem cell. Methods are provided for the isolation and culture of these cells. The cell enrichment methods employ reagents that specifically recognize CD34, CD38, CD90 and CD45RA, in conjunction with lineage specific markers. These cells give rise to all types of hematopoietic cells, e.g. myeloid and lymphoid cells, in vivo.

### GOVERNMENT RIGHTS

This invention was made with Government support under contract CA086017 awarded by the National Institutes of Health. The Government has certain rights in this invention.

### BACKGROUND OF THE INVENTION

Hematopoiesis proceeds through an organized developmental hierarchy initiated by hematopoietic stem cells (HSC) that give rise to progressively more committed progenitors and eventually terminally differentiated blood cells. Stem cells are defined as cells that have the ability to perpetuate themselves through self-renewal and to generate mature cells of a particular tissue through differentiation. Perhaps the most important and useful property of stem cells is that of self-renewal. Like stem cells, progenitor cells generate mature cells of a particular tissue through differentiation, however by conventional usage the term “progenitor” refers to a cell that is not maintained for extended periods of time through self-renewal.

Although the concept of the HSC was not new, it was not until 1988 that it was shown that this population could be prospectively isolated from mouse bone marrow on the basis of cell-surface markers using fluorescence-activated cell sorting (FACS). Since that time, the surface immunophenotype of the mouse HSC has become increasingly refined, such that functional HSC can be isolated with exquisite sensitivity, resulting in a purity of 1 in 1.3 cells.

Both in vitro and in vivo experimental approaches have also been utilized to identify human HSC. The best in vitro assay of HSC activity is the long-term culture-initiating cell assay (LTC-IC), which requires culturing of cells on bone marrow feeder cells to identify those capable of producing hematopoietic cells for 6 weeks or longer. Using this technique, candidate human HSC were identified by virtue of expression of CD34 and CD90 (Thy1) and lack of lineage markers (Lin⁻) (see Baum et al. (1992) Proc Natl Acad Sci U S A 89, 2804-2808; Craig et al. (1993) J Exp Med 177, 1331-1342). Other studies using the LTC-IC assay localized human HSC activity to the Lin⁻CD34⁺CD38^{-/lo} fraction (Kondo et al. (2003) Annu Rev Immunol 21, 759-806).

Although these in vitro assays provide important information regarding lineage potential and possibly self-renewal, it is clear that definitive demonstration of HSC function requires an in vivo assay. Several in vivo models have been utilized to study human hematopoiesis. McCune and colleagues used the SCID-hu mouse model to identify human HSC activity among Lin−CD34+CD90+cells (see McCune et al. (1988) Science 241, 1632-1639; Murray et al. (1995) Blood 85, 368-378).

Dick and colleagues initially used SCID/beige/XID and, more recently, NOD/SCID mice to assay normal human progenitor subpopulations Dick et al. (2001) Ann N Y Acad Sci 938, 184-190. By assessing long-term multipotent human hematopoiesis in recipients and the ability to form secondary and tertiary transplants, human HSC were found to reside in the Lin−CD34+CD38−/lo fraction of human progenitors (Hogan et al. (2002) Proc Natl Acad Sci U S A 99, 413-418). Recently, the NOD/SCID/IL-2Rγ null strain (NOG) strain was shown to exhibit significantly higher engraftment potential than other immunodeficient mouse strains when transplanted with human hematopoietic progenitors. In vivo demonstration of HSC function also comes from human trials of autologous mobilized peripheral blood in clinical transplantation, where long-term engraftment was provided by transplantation of purified CD34+CD90+ cells (Negrin et al. (2000) Biol Blood Marrow Transplant 6, 262-271). Together, these in vivo studies support the idea that human HSC are contained in the Lin−CD34+CD38−CD90+ fraction of hematopoietic cells.

HSC are defined on the basis of two key properties: (1) multipotency, defined as the ability to form all differentiated blood cells, and (2) long-term self-renewal, defined as the lifelong ability to give rise to progeny identical to the parent through cell division. HSC are the only hematopoietic cells that possess these two properties; however, in the mouse hematopoietic cells have been identified that are multipotent, but not capable of long-term self-renewal, termed multipotent progenitors (MPP). MPP lie immediately downstream of HSC within the hematopoietic hierarchy and have been identified in mouse bone marrow on the basis of their distinct surface immunophenotype (Christensen and Weissman (2001) Proc Natl Acad Sci U S A 98, 14541-14546; Morrison et al. (1997) Development 124, 1929-1939).

MPP have yet to be identified within the human hematopoiesis hierarchy. It has been observed that retroviral or lentiviral marking of Lin−CD34+CD38−/lo cells prior to xenotransplantation allowed investigators to track engrafted cells, resulting in the observation that individual Lin−CD34+CD38−/lo cells had variable self-renewal and proliferation potential (McKenzie et al. (2006) Nat Immunol 7, 1225-1233), although it was not determined if the short-term clones were multipotent.

The isolation and characterization of cells in the human hematopoietic lineage are of great interest for clinical and research uses. The present invention identified a new multipotent progenitor cell.

### SUMMARY OF THE INVENTION

A substantially enriched human hematopoietic cell population is provided, which is characterized as a multipotent progenitor cell (MPP) committed to the hematopoietic lineage, which is able to give rise to all differentiated hematopoietic cells, but which lacks certain long-term self-renewal properties of the hematopoietic stem cell. In vivo, MPPs give rise to differentiated cells for at least about 4 weeks. The MPP cells are useful in transplantation, for experimental evaluation, and as a source of lineage and cell specific products, including mRNA species useful in identifying genes specifically expressed in these cells, and as targets for the discovery of factors or molecules that can affect them.

Selection for expression of cell surface markers is used to separate the MPP cells from more differentiated cells and from HSC. The Lin−CD34+CD38− fraction of hematopoietic cells is shown to be subdivided into three subpopulations: CD90+CD45RA−, CD90−CD45RA−, and CD90−CD45RA+. Utilizing in vivo transplantation studies and complementary in vitro assays, it is demonstrated that the Lin−CD34+CD38−CD90+CD45RA− fraction contains HSC; and human multipotent progenitor cells are found within the Lin−CD34+CD38−CD90−CD45RA− fraction.

### BRIEF DESCRIPTION OF THE DRAWINGS

FIG. 1: Identification of CD90/CD45RA Subpopulations of Lin−CD34+CD38− Human Bone Marrow and Cord Blood. Normal human bone marrow (top) and cord blood (bottom) were analyzed for expression of lineage markers, CD34, CD38, CD90, and CD45RA by flow cytometry. The bone marrow sample was CD34-enriched prior to analysis. The left panels are gated on lineage negative (Lin−) live cells, while the right panels are gated on Lin−CD34+CD38− cells. Data shown is representative of multiple samples of bone marrow (n=10) and cord blood (n=22).

FIG. 2: In Vitro Evaluation of the CD90/CD45RA Subpopulations of Lin−CD34+CD38− Cord Blood Reveals a Developmental Hierarchy. A. Methylcellulose colony formation. Single cells from each CD90/CD45RA subpopulation were sorted into individual wells of a 96 well plate containing complete methylcellulose capable of supporting growth of all types of myeloid colonies. After 12-14 days, colonies were scored based on morphology. The percent of each type of colony out of the total cells plated is indicated. Data presented is cumulative from 3 experiments of 60 cells each, for a total of 180 cells per subpopulation. B. Methylcellulose colony replating. All colonies derived from individual cells were harvested, dissociated, and replated in complete methylcellulose. 12-14 days later, the formation of new colonies was scored based on morphology. 42 out of 62 (70%) of CD90+CD45RA−, 23 out of 70 (33%) of CD90−CD45RA−, and 0 out of 6 (0%) of CD90−CD45RA+ colonies formed new colonies upon replating. The difference in replating efficiency between CD90+CD45RA− and CD90−CD45RA− was statistically significant (p=0.003). Data presented is the average of 3 independent experiments with the indicated SEM. C. In vitro proliferation. 20 cells of each CD90/CD45RA subpopulation were clone sorted into individual wells of a 96 well plate containing serum-free media supplemented with human LDL and cytokines. After 2 weeks in culture, cells were harvested and live cells counted by trypan blue exclusion. The difference between the CD90+CD45RA− and the CD90−CD45RA− subpopulations was statistically significant (p=0.007). Data is representative of 3 independent experiments. D. In vitro hierarchical relationships among CD90/CD45RA subpopulations. CD90/CD45RA subpopulations were sorted in bulk into serum-free media supplemented with human LDL and cytokines. Cells were cultured for four days and then re-analyzed by flow cytometry. All plots shown are gated on Lin−CD34+CD38− cells; the left panels show the cell input; the right panels show the cells after four days in culture. Data shown is representative of 3 independent experiments.

FIG. 3: Long-Term In Vivo Multipotent Human Hematopoiesis with Transplantation of as few as 10 Lin−CD34+CD38−CD90+CD45RA− Cord Blood Cells. A. In vivo engraftment of 100 or 10 CD90+CD45RA− cells. 100 or 10 FACS-purified CD90+CD45RA− cells were transplanted into NOG newborn mice as described. 12 weeks later peripheral blood and/or bone marrow was harvested and analyzed by flow cytometry for the presence of human CD45+ hematopoietic cells, myeloid cells (hCD45+CD13+), and B lymphoid cells (hCD45+CD19+) cells. The right plots are gated on human CD45+ cells. B. Wright-Giemsa stained cytospin preparations from CD90+CD45RA− engrafted mice. Human CD45+ cells were purified by FACS from peripheral blood (panels 1-3) or bone marrow (panel 4) of mice engrafted with CD90+CD45RA− cells. In the peripheral blood, (1) lymphocytes, (2) neutrophils, and (3) monocytes were detected; in the bone marrow (4) lymphocytes and maturing myeloid cells were detected. (100×)

FIG. 4: Human Lymphoid and Myeloid Cells Reconstitute the Peripheral Blood of CD90/CD45RA Transplanted Mice. A. Peripheral blood engraftment and lineage analysis of CD90/CD45RA transplanted mice. 500 FACS-purified cells of each CD90/CD45RA subpopulation: CD90+CD45RA− (top panels), CD90−CD45RA− (middle panels), and CD90−CD45RA+ (bottom panels), were transplanted into NOG newborn mice as described. 12 weeks later peripheral blood was harvested and analyzed by flow cytometry for the presence of human CD45+ hematopoietic cells, myeloid cells (hCD45+CD13+), and B lymphoid cells (hCD45+CD19+) cells. The right plots are gated on human CD45+ cells. B. Summary of long-term (>12 weeks) peripheral blood engraftment of CD90/CD45RA subpopulations. C. Peripheral blood engraftment per 100 transplanted cells. The percent human chimerism (left) and percent human myeloid cells (right) per 100 transplanted cells is indicated for each engrafted mouse. Each circle or triangle represents an individual mouse and the bar indicates the average. On average, CD90+CD45RA− mice developed 7 fold more human chimerism than CD90−CD45RA− mice, and this difference was statistically significant (p=0.02). The 7 fold difference in percent human myeloid cells approached, but did not achieve statistical significance (p=0.08).

FIG. 5: Human Lymphoid and Myeloid Cells Reconstitute the Bone Marrow of CD90+CD45RA− Transplanted Mice More Efficiently than CD90−CD45RA− Transplanted Mice. A. Bone marrow engraftment and lineage analysis of CD90/CD45RA transplanted mice. 500 FACS-purified CD90+CD45RA− cells (top panels) and CD90−CD45RA− cells (bottom panels) were transplanted into NOG newborn mice as described. 12 weeks later bone marrow was analyzed by flow cytometry for the presence of human CD45+ hematopoietic cells, myeloid cells (hCD45+CD33+), and B lymphoid cells (hCD45+CD19+) cells. The right plots are gated on human CD45+ cells. B. Summary of long-term (>12 weeks) bone marrow engraftment of CD90/CD45RA subpopulations. C. Bone marrow engraftment per 100 transplanted cells. The percent human chimerism (left) and percent human myeloid cells (right) per 100 transplanted cells is indicated for each engrafted mouse. Each circle or triangle represents an individual mouse and the bar indicates the average. On average, CD90+CD45RA− mice developed 9 fold more human chimerism than CD90−CD45RA− mice, and this difference was statistically significant (p=0.001). The 9 fold difference in percent human myeloid cells approached, but did not achieve statistical significance (p=0.07). D. Summary of long-term (>11 weeks) bone marrow engraftment of limiting numbers (<100 cells) of CD90/CD45RA subpopulations. 50 or 70 double FACS-purified CD90+CD45RA− or CD90−CD45RA− cells were transplanted into NOG newborn mice as described. At least 11 weeks later bone marrow was analyzed by flow cytometry for the presence of human CD45+ hematopoietic cells, myeloid cells (hCD45+CD33+), B cells (hCD45+CD19+) cells, and T cells (hCD45+CD3+). In 1 out of 5 mice transplanted with CD90−CD45RA− cells, a small population of T cells (0.2%) was detected in the bone marrow, in the absence of myeloid and B cells. The difference in successful engraftment was statistically significant (p=0.008).

FIG. 6: In Vivo Analysis of Human CD34+ Cells Identifies a Hierarchy Among the CD90/CD45RA Subpopulations. A. Summary of long-term (>12 weeks) human CD34+ bone marrow engraftment of CD90/CD45RA subpopulations. B. Bone marrow human CD34+ engraftment per 100 transplanted cells. The percentage of human CD34+ cells in total bone marrow per 100 transplanted cells is indicated for each engrafted mouse. Each circle represents an individual mouse and the bar indicates the average. On average, CD90+CD45RA− mice contained 8 fold more human CD34+ cells than CD90−CD45RA− mice, and this difference was statistically significant (p=0.01). C. Analysis of CD90/CD45RA expression on Lin−CD34+CD38−bone marrow cells in CD90/CD45RA transplanted mice. 500 FACS-purified CD90+CD45RA− cells (top panels) and CD90−CD45RA− cells (bottom panels) were transplanted into NOG newborn mice as described. 12 weeks later bone marrow was analyzed by flow cytometry for the expression of lineage markers, CD34, CD38, CD90, and CD45RA. The left plots are gated on Lin−CD34+ cells, and the right plots are gated on Lin−CD34+CD38− cells. D. CD90/CD45RA subpopulations within engrafted bone marrow. The percentage of CD90+CD45RA− (left), CD90−CD45RA− (middle), and CD90−CD45RA+ (right) cells out of Lin−CD34+CD38− bone marrow cells from mice engrafted with CD90+CD45RA− and CD90−CD45RA− cells is indicated. Each circle, triangle, or square represents an individual mouse. Only mice with greater than 10 Lin−CD34+CD38− cells were included (n=7 transplanted with CD90+CD45RA− cells and n=4 transplanted with CD90−CD45RA− cells).

FIG. 7: Enrichment of Secondary Transplant Ability in the CD90+CD45RA Subpopulation. A. Summary of long-term (>10 weeks) bone marrow engraftment in secondary transplants from mice engrafted with either CD90+ or CD90− subpopulations. The difference in successful secondary engraftment, 12 of 12 (100%) for CD90+ versus 3 of 8 (37.5%) for CD90−, was statistically significant with p=0.004. B. Bone marrow engraftment in secondary recipients from experiment 1. Primary mice were transplanted with 2000 cells of the indicated population. The percent human chimerism (left) and percent myeloid cells of total human CD45+ cells (right) is indicated for each secondary mouse. Each circle or triangle represents an individual mouse. C. Bone marrow engraftment in secondary recipients from experiment 2. Primary mice were transplanted with 500 cells of the indicated population. The percent human chimerism (left) and percent myeloid cells of total human CD45+ cells (right) is indicated for each secondary mouse. Each circle or triangle represents an individual mouse.

FIG. 8. Purification of CD90/CD45RA Subpopulations of Lin−CD34+CD38− Cord Blood by FACS. Normal human cord blood was analyzed for expression of lineage markers, CD34, CD38, CD90, and CD45RA. The pre-sorting profile of CD90and CD45RA expression on Lin−CD34+CD38− cells is shown in the top panel. The indicated CD90/CD45RA subpopulations were isolated by FACS and re-analyzed for purity. Each population was successfully isolated to >95% purity. Cells in each plot are gated on Lin−CD34+CD38− live cells. Data shown is representative of >10 purifications of independent cord blood samples.

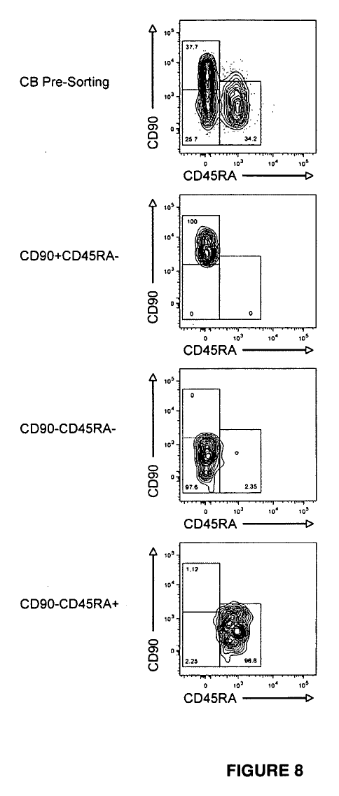

FIG. 9. Human Lymphoid Splenic Engraftment in CD90/CD45RA Transplanted Mice. A. Summary of long-term (>12 weeks) spleen engraftment of CD90/CD45RA subpopulations. B. Spleen engraftment per 100 transplanted cells. The percent human chimerism (left) and percent T cells of total human CD45+ cells (right) per 100 transplanted cells is indicated for each engrafted mouse. Each circle or triangle represents an individual mouse and the bar indicates the average. On average, CD90+CD45RA− mice developed 7 fold more human chimerism than CD90−CD45RA− mice, and this difference was statistically significant (p=0.025). No statistically significant difference was detected in the percent T cells of total human CD45+ cells.

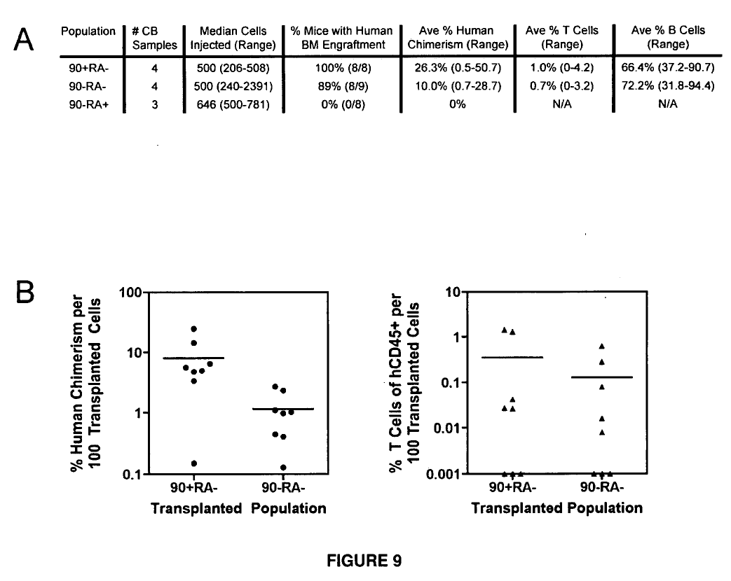

FIG. 10. Total Engraftment Data of CD90+CD45RA− and CD90−CD45RA− Subpopulations. A. Summary of long-term (>12 weeks) peripheral blood engraftment. B. Summary of long-term (>12 weeks) bone marrow engraftment. C. Summary of long-term (>12 weeks) spleen engraftment. D. Summary of long-term (>12 weeks) secondary transplant bone marrow engraftment.

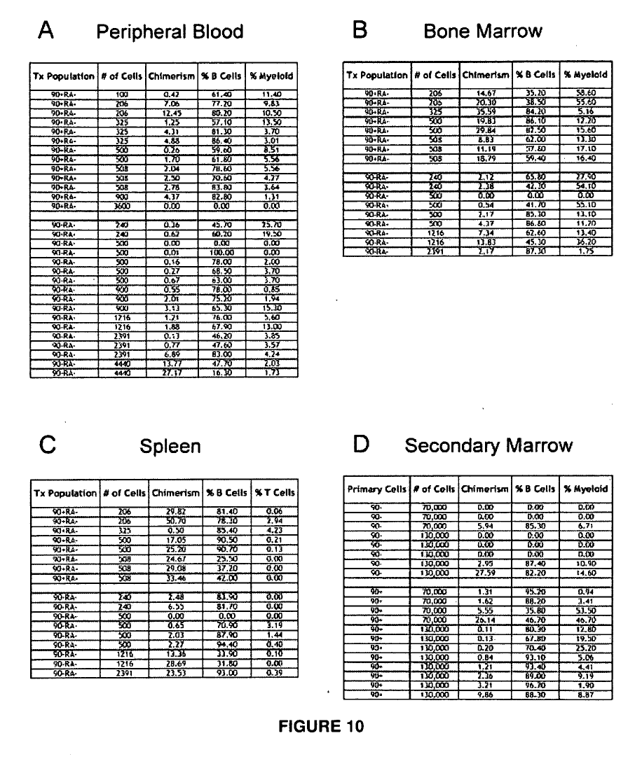

FIG. 11. Multipotent Populations in Mouse and Human Hematopoiesis. Hematopoiesis is organized as a hierarchy originating with hematopoietic stem cells (HSC) that in turn give rise to downstream multipotent progenitors (MPP). HSC and MPP differ in their ability to self-renew, as HSC are the only cells capable of lifelong self-renewal. The surface immunophenotype is indicated for each mouse and human population, where identified. Note that in human cord blood the Lin−CD34+CD38−CD90−CD45RA+ population lies downstream from the Lin−CD34+CD38−CD90−CD45RA− multipotent progenitor.

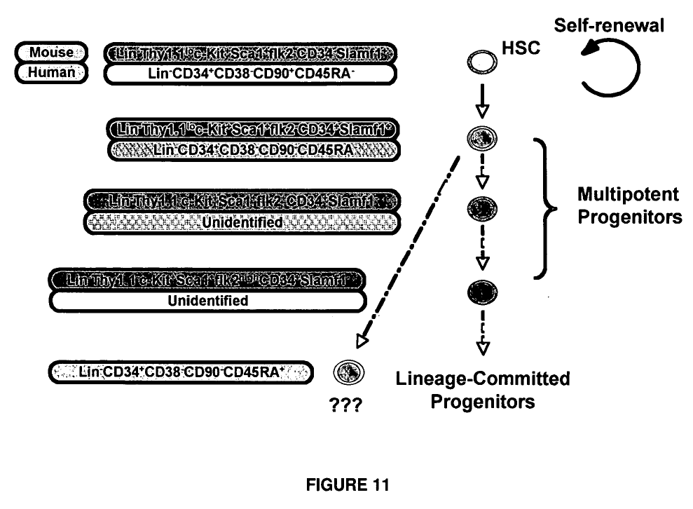

### DETAILED DESCRIPTION OF THE EMBODIMENTS

A substantially enriched human multipotent progenitor cell (MPP) population is provided. The cells are useful in generating differentiated cells of the hematopoietic lineages for defined periods of time. The MPP cells are useful in transplantation, for experimental evaluation, and as a source of lineage and cell specific products. In some embodiments, the cells are defined as being Lin−CD34+CD38−CD90−CD45RA−, where the lineage panel may include CD2; CD3; CD4; CD7; CD8; CD10; CD11b; CD14; CD19; CD20; CD56; and glycophorin A (GPA).

The term “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “individual,” “subject,” “host,” and “patient,” used interchangeably herein and refer to any mammalian subject for whom diagnosis, treatment, or therapy is desired, particularly humans.

A “host cell”, as used herein, refers to a microorganism or a eukaryotic cell or cell line cultured as a unicellular entity which can be, or has been, used as a recipient for a recombinant vector or other transfer polynucleotides, and include the progeny of the original cell which has been transfected. It is understood that the progeny of a single cell may not necessarily be completely identical in morphology or in genomic or total DNA complement as the original parent, due to natural, accidental, or deliberate mutation.

The terms “cancer”, “neoplasm”, “tumor”, and “carcinoma”, are used interchangeably herein to refer to cells which exhibit relatively autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. Detection of cancerous cells is of particular interest. The term “normal” as used in the context of “normal cell,” is meant to refer to a cell of an untransformed phenotype or exhibiting a morphology of a non-transformed cell of the tissue type being examined. “Cancerous phenotype” generally refers to any of a variety of biological phenomena that are characteristic of a cancerous cell, which phenomena can vary with the type of cancer. The cancerous phenotype is generally identified by abnormalities in, for example, cell growth or proliferation (e.g., uncontrolled growth or proliferation), regulation of the cell cycle, cell mobility, cell-cell interaction, or metastasis, etc.

“Therapeutic target” refers to a gene or gene product that, upon modulation of its activity (e.g., by modulation of expression, biological activity, and the like), can provide for modulation of the cancerous phenotype. As used throughout, “modulation” is meant to refer to an increase or a decrease in the indicated phenomenon (e.g., modulation of a biological activity refers to an increase in a biological activity or a decrease in a biological activity).

Mammalian multipotent hematopoietic progenitor cells are provided, herein termed MPP. The MPP population is useful in transplantation; for drug screening; experimental models of hematopoietic differentiation and interaction; screening in vitro assays to define growth and differentiation factors, and to characterize genes involved in hematopoietic development and regulation; and the like. The native cells may be used for these purposes, or they may be genetically modified to provide altered capabilities.

The multipotent progenitor cells can be separated from a complex mixture of cells by using reagents that specifically recognize markers on the cell surface. The MPP cells express detectable levels of the marker CD34, and are selected for a lack of expression of the markers Thy-1 (CD90), CD38, CD45RA; and/or with a lineage panel of markers, as further described below.

### Phenotypic Characterization

It will be understood by those of skill in the art that the stated expression levels reflect detectable amounts of the marker protein on the cell surface. A cell that is negative for staining (the level of binding of a marker specific reagent is not detectably different from an isotype matched control) may still express minor amounts of the marker. And while it is commonplace in the art to refer to cells as “positive” or “negative” for a particular marker, actual expression levels are a quantitative trait. The number of molecules on the cell surface can vary by several logs, yet still be characterized as “positive”.

The staining intensity of cells can be monitored by flow cytometry, where lasers detect the quantitative levels of fluorochrome (which is proportional to the amount of cell surface marker bound by specific reagents, e.g. antibodies). Flow cytometry, or FACS, can also be used to separate cell populations based on the intensity of binding to a specific reagent, as well as other parameters such as cell size and light scatter. Although the absolute level of staining may differ with a particular fluorochrome and reagent preparation, the data can be normalized to a control.

In order to normalize the distribution to a control, each cell is recorded as a data point having a particular intensity of staining. These data points may be displayed according to a log scale, where the unit of measure is arbitrary staining intensity. In one example, the brightest stained cells in a sample can be as much as 4 logs more intense than unstained cells. When displayed in this manner, it is clear that the cells falling in the highest log of staining intensity are bright, while those in the lowest intensity are negative. The “low” positively stained cells have a level of staining above the brightness of an isotype matched control, but is not as intense as the most brightly staining cells normally found in the population. An alternative control may utilize a substrate having a defined density of marker on its surface, for example a fabricated bead or cell line, which provides the positive control for intensity.

### Markers

The MPP cells are positive for CD34 expression. CD34 is a monomeric cell surface antigen with a molecular mass of approximately 110 kD that is selectively expressed on human hematopoietic progenitor cells. The gene is expressed by small vessel endothelial cells in addition to hematopoietic progenitor cells and is a single-chain 105-120 kDa heavily O-glycosylated transmembrane glycoprotein. The sequence is disclosed by Simmons et al. (1992) J. Immun. 148:267-271. Antibodies are commercially available, for example from BD Biosciences, Pharmingen, San Diego, Calif., catalog number 550760.

The MPP cells are negative for expression of CD38. CD38 is a 300-amino acid type II transmembrane protein with a short N-terminal cytoplasmic tail and 4 C-terminal extracellular N-glycosylation sites. The sequence is disclosed by Jackson et al. (1990) J. Immun. 144: 2811-2815. The marker is generally associated with lymphocytes, myeloblasts, and erythroblasts. Antibodies are commercially available, for example from BD Biosciences, Pharmingen, San Diego, Calif., catalog number 347680.

The MPP cells have the phenotype of lacking expression of lineage specific markers. For staining purposes a cocktail of binding reagents, herein designated “lin”, may be used. The lin panel will comprise binding reagents, e.g. antibodies and functional binding fragments thereof, ligands, peptidomimetics, etc., that recognize two or more of the lineage markers. A lin panel will generally include at least one marker expressed on mature B cells, on mature T cells, on mature granulocytes and on mature macrophages. Markers suitable for use in a lineage panel are typically expressed on these mature cells, but are not present on multiple lineages, or on stem and progenitor cells. Lineage markers may include CD2; CD3; CD4; CD7; CD8; CD10; CD11b; CD14; CD19; CD20; CD56; and glycophorin A (GPA) in humans. The MPP cells are negative for expression of Thy-1 (CD90), which is a 25-35 kD molecule expressed on 1-4% of human fetal liver cells, cord blood cells, and bone marrow cells. Antibodies are commercially available, for example from BD Biosciences, Pharmingen, San Diego, Calif., catalog number 555595.

### Methods of Enrichment

Methods for enrichment of MPP cells are provided. The enriched cell population will usually have at least about 80% cells of the selected phenotype, more usually at least 90% cells and may be 95% of the cells, or more, of the selected phenotype. The subject cell populations are separated from other cells, e.g. hematopoietic cells, on the basis of specific markers, which are identified with affinity reagents, e.g. monoclonal antibodies.

Ex vivo and in vitro cell populations useful as a source of cells may include fresh or frozen cells, which may be fetal, neonatal, juvenile or adult, including bone marrow, spleen, liver, umbilical cord blood, peripheral blood, mobilized peripheral blood, yolk sac, etc. For autologous or allogeneic transplantation, bone marrow and mobilized peripheral blood are preferred starting materials. For peripheral blood, progenitor cells are mobilized from the marrow compartment into the peripheral bloodstream after treatment with chemotherapy; G-CSF or GM-CSF, or both. A number of single and combination chemotherapeutic agents have been used to mobilize cells. A review of peripheral blood stem cells may be found in Shpall et al. (1997) Annu Rev Med 48:241-251, and the characterization of stem cell mobilization in Moog et al. (1998) Ann Hematol 77(4):143-7. As an alternative source of cells, hematopoietic stem cells, as described in U.S. Patent No. 5,061,620, issued on Oct. 29, 1991; and U.S. Pat. No. 5,087,570, issued Feb. 11, 1992, may be cultured and induced to differentiate in vivo or in vitro to provide a source of cells.

The progenitor cells may be obtained from any mammalian species, e.g. human, equine, bovine, porcine, canine, feline, rodent, e.g. mice, rats, hamster, primate, etc. The tissue may be obtained by biopsy or aphoresis from a live donor, or obtained from a dead or dying donor within about 48 hours of death, or freshly frozen tissue, tissue frozen within about 12 hours of death and maintained at below about −20° C., usually at about liquid nitrogen temperature (−180° C.) indefinitely.

The subject cells are separated from a complex mixture of cells by techniques that enrich for cells that express certain cell surface markers, while lacking certain cell specific markers, for example by initially selecting for cells that are Lin−CD34+CD38−, and then selecting for the population that is CD90−CD45RA−. Alternatively, selection may be made for all markers simultaneously, or for any suitable sequential process, e.g. performing a negative selection, e.g. for one or more of lineage markers, CD38, CD90and CD45RA; followed by a positive selection for CD34.

For isolation of cells from tissue, an appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

Separation of the subject cell populations will then use affinity separation to provide a substantially pure population. Techniques for affinity separation may include magnetic separation, using antibody-coated magnetic beads, affinity chromatography, cytotoxic agents joined to a monoclonal antibody or used in conjunction with a monoclonal antibody, e.g. complement and cytotoxins, and “panning” with antibody attached to a solid matrix, eg. plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide). Any technique may be employed which is not unduly detrimental to the viability of the selected cells.

The affinity reagents may be specific receptors or ligands for the cell surface molecules indicated above. In addition to antibody reagents, peptide-MHC antigen and T cell receptor pairs may be used; peptide ligands and receptor; effector and receptor molecules, and the like. Antibodies and T cell receptors may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art.

Of particular interest is the use of antibodies as affinity reagents. Conveniently, these antibodies are conjugated with a label for use in separation. Labels include magnetic beads, which allow for direct separation, biotin, which can be removed with avidin or streptavidin bound to a support, fluorochromes, which can be used with a fluorescence activated cell sorter, or the like, to allow for ease of separation of the particular cell type. Fluorochromes that find use include phycobiliproteins, e.g. phycoerythrin and allophycocyanins, fluorescein and Texas red. Frequently each antibody is labeled with a different fluorochrome, to permit independent sorting for each marker.

The antibodies are added to a suspension of cells, and incubated for a period of time sufficient to bind the available cell surface antigens. The incubation will usually be at least about 5 minutes and usually less than about 30 minutes. It is desirable to have a sufficient concentration of antibodies in the reaction mixture, such that the efficiency of the separation is not limited by lack of antibody. The appropriate concentration is determined by titration. The medium in which the cells are separated will be any medium that maintains the viability of the cells. A preferred medium is phosphate buffered saline containing from 0.1 to 0.5% BSA. Various media are commercially available and may be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, etc.

The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum.

Compositions highly enriched for multipotent progenitor activity are achieved in this manner. The subject population will be at or about 80% or more of the cell composition, and preferably be at or about 95% or more of the cell composition. The desired cells are identified by their surface phenotype, by the ability to respond to growth factors, and being able to provide for development in vivo and in vitro of all mature hematopoietic cells. The enriched cell population may be used immediately, or may be frozen at liquid nitrogen temperatures and stored for long periods of time, being thawed and capable of being reused. The cells will usually be stored in 10% DMSO, 50% FCS, 40% RPMI 1640 medium. Once thawed, the cells may be expanded by use of growth factors or stromal cells associated with hematopoietic cell proliferation and differentiation.

### In Vitro Culture

The enriched cell population may be grown in vitro under various culture conditions. Culture medium may be liquid or semi-solid, e.g. containing agar, methylcellulose, etc. The cell population may be conveniently suspended in an appropriate nutrient medium, such as Iscove's modified DMEM or RPMI-1640, normally supplemented with fetal calf serum (about 5-10%), L-glutamine, a thiol, particularly 2-mercaptoethanol, and antibiotics, e.g. penicillin and streptomycin.

The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

In addition to, or instead of growth factors, the subject cells may be grown in a co-culture with stromal or feeder layer cells. Stromal cells suitable for use in the growth of hematopoietic cells are known in the art. These include bone marrow stroma as used in “Whitlock-Witte” (Whitlock et al. [1985] Annu Rev Immunol 3:213-235) or “Dexter” culture conditions (Dexter et al. [1977] J Exp Med 145:1612-1616); and heterogeneous thymic stromal cells (Small and Weissman [1996] Scand J Immunol 44:115-121).

The subject cultured cells may be used in a wide variety of ways. The nutrient medium, which is a conditioned medium, may be isolated at various stages and the components analyzed. Separation can be achieved with HPLC, reversed phase-HPLC, gel electrophoresis, isoelectric focusing, dialysis, or other non-degradative techniques, which allow for separation by molecular weight, molecular volume, charge, combinations thereof, or the like. One or more of these techniques may be combined to enrich further for specific fractions.

Genes may be introduced into the MPP cells for a variety of purposes, e.g. replace genes having a loss of function mutation, markers, etc. Alternatively, vectors are introduced that express antisense mRNA or ribozymes, thereby blocking expression of an undesired gene. Other methods of gene therapy are the introduction of drug resistance genes to enable normal progenitor cells to have an advantage and be subject to selective pressure, for example the multiple drug resistance gene (MDR), or anti-apoptosis genes, such as bcl-2. Various techniques known in the art may be used to transfect the target cells, e.g. electroporation, calcium precipitated DNA, fusion, transfection, lipofection and the like. The particular manner in which the DNA is introduced is not critical to the practice of the invention.

Many vectors useful for transferring exogenous genes into target mammalian cells are available. The vectors may be episomal, e.g. plasmids, virus derived vectors such cytomegalovirus, adenovirus, etc., or may be integrated into the target cell genome, through homologous recombination or random integration, e.g. retrovirus derived vectors such MMLV, HIV-1, ALV, etc. Retrovirus based vectors have been shown to be particularly useful when the target cells are hematopoietic progenitor cells. For example, see Schwarzenberger et al. (1996) Blood 87:472-478; Nolta et al. (1996) P.N.A.S. 93:2414-2419; and Maze et al. (1996) P.N.A.S. 93:206-210.

Combinations of retroviruses and an appropriate packaging line may be used, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, ie. unable to produce viral proteins required for productive infection. Replication of the vector requires growth in the packaging cell line. Lentiviral vectors such as those based on HIV or FIV gag sequences can be used to transfect non-dividing cells, such as the resting phase of human long term hematopoietic stem cells (see Uchida et al. (1998) P.N.A.S. 95(20):11939-44).

The host cell specificity of the retrovirus is determined by the envelope protein, env (p120). The envelope protein is provided by the packaging cell line. Envelope proteins are of at least three types, ecotropic, amphotropic and xenotropic. Retroviruses packaged with ecotropic envelope protein, e.g. MMLV, are capable of infecting most murine and rat cell types. Ecotropic packaging cell lines include BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse. Amphotropic packaging cell lines include PA12 (Miller et al. (1985) Mol. Cell. Biol. 5:431 437); PA317 (Miller et al. (1986) Mol. Cell. Biol. 6:2895-2902) GRIP (Danos et al. (1988) PNAS 85:6460□6464). Retroviruses packaged with xenotropic envelope protein, e.g. AKR env, are capable of infecting most mammalian cell types, except murine cells.

The sequences at the 5′ and 3′ termini of the retrovirus are long terminal repeats (LTR). A number of LTR sequences are known in the art and may be used, including the MMLV-LTR; HIV-LTR; AKR-LTR; FIV-LTR; ALV-LTR; etc. Specific sequences may be accessed through public databases. Various modifications of the native LTR sequences are also known. The 5′ LTR acts as a strong promoter, driving transcription of the introduced gene after integration into a target cell genome. For some uses, however, it is desirable to have a regulatable promoter driving expression. Where such a promoter is included, the promoter function of the LTR will be inactivated. This is accomplished by a deletion of the U3 region in the 3′LTR, including the enhancer repeats and promoter, which is sufficient to inactivate the promoter function. After integration into a target cell genome, there is a rearrangement of the 5′ and 3′ LTR, resulting in a transcriptionally defective provirus, termed a “self-inactivating vector”. The vectors may include genes that must later be removed, e.g. using a recombinase system such as Cre/Lox, or the cells that express them destroyed, e.g. by including genes that allow selective toxicity such as herpesvirus TK, bcl-xs, etc. Suitable inducible promoters are activated in a desired target cell type, either the transfected cell, or progeny thereof. By transcriptional activation, it is intended that transcription will be increased above basal levels in the target cell by at least about 100 fold, more usually by at least about 1000 fold. Various promoters are known that are induced in hematopoietic cell types.

To prove that one has genetically modified progenitor cells, various techniques may be employed. The genome of the cells may be restricted and used with or without amplification. The polymerase chain reaction; gel electrophoresis; restriction analysis; Southern, Northern, and Western blots; sequencing; or the like, may all be employed. The cells may be grown under various conditions to ensure that the cells are capable of maturation to all of the myeloid lineages while maintaining the ability to express the introduced DNA. Various tests in vitro and in vivo may be employed to ensure that the pluripotent capability of the cells has been maintained.

### Transplantation

The subject MPP cells may be used for reconstitution or supplementing lymphoid, megakaryocytic, erythroid, and myeloid cells in a recipient. A need for transplantation may be caused by genetic or environmental conditions, e.g. chemotherapy, exposure to radiation, etc. Autologous cells, particularly if removed prior to cytoreductive or other therapy, or allogeneic cells, may be used for progenitor cell isolation and subsequent transplantation.

The progenitor cells may be administered in any physiologically acceptable medium, normally intravascularly, although they may also be introduced into bone or other convenient site, where the cells may find an appropriate site for regeneration and differentiation. Usually, at least 1×10⁵ cells will be administered, preferably 1×10⁶ or more. The cells may be introduced by injection, catheter, or the like. The cells may be frozen at liquid nitrogen temperatures and stored for long periods of time, being capable of use on thawing. If frozen, the cells will usually be stored in a 10% DMSO, 50% FCS, 40% RPMI 1640 medium. Once thawed, the cells may be expanded by use of growth factors and/or stromal cells associated with progenitor cell proliferation and differentiation.

### Screening Methods

The subject cells are useful for in vitro assays and screening to detect factors that are active on multipotent progenitors. A wide variety of assays may be used for this purpose, including immunoassays for protein binding; determination of cell growth, differentiation and functional activity; and the like.

Also of interest is the examination of gene expression in the MPP cells. The expressed set of genes may be compared between the progenitors and mature hematopoietic cells or subsets of cells, as known in the art. Of particular interest is the comparison of MPP cells with acute myelocytic leukemia AML) cells.

Any suitable qualitative or quantitative methods known in the art for detecting specific mRNAs can be used. mRNA can be detected by, for example, hybridization to a microarray, in situ hybridization in tissue sections, by reverse transcriptase-PCR, or in Northern blots containing poly A+ mRNA. One of skill in the art can readily use these methods to determine differences in the size or amount of mRNA transcripts between two samples. For example, the level of particular mRNAs in MPP cells is compared with the expression of the mRNAs in a reference sample, e.g. CMP cells.

Any suitable method for detecting and comparing mRNA expression levels in a sample can be used in connection with the methods of the invention. For example, mRNA expression levels in a sample can be determined by generation of a library of expressed sequence tags (ESTs) from a sample. Enumeration of the relative representation of ESTs within the library can be used to approximate the relative representation of a gene transcript within the starting sample. The results of EST analysis of a test sample can then be compared to EST analysis of a reference sample to determine the relative expression levels of a selected polynucleotide, particularly a polynucleotide corresponding to one or more of the differentially expressed genes described herein.

Alternatively, gene expression in a test sample can be performed using serial analysis of gene expression (SAGE) methodology (Velculescu et al., Science (1995) 270:484). In short, SAGE involves the isolation of short unique sequence tags from a specific location within each transcript. The sequence tags are concatenated, cloned, and sequenced. The frequency of particular transcripts within the starting sample is reflected by the number of times the associated sequence tag is encountered with the sequence population.

Gene expression in a test sample can also be analyzed using differential display (DD) methodology. In DD, fragments defined by specific sequence delimiters (e.g., restriction enzyme sites) are used as unique identifiers of genes, coupled with information about fragment length or fragment location within the expressed gene. The relative representation of an expressed gene with a sample can then be estimated based on the relative representation of the fragment associated with that gene within the pool of all possible fragments. Methods and compositions for carrying out DD are well known in the art, see, e.g., U.S. Pat. No. 5,776,683; and U.S. Pat. No. 5,807,680.

Alternatively, gene expression in a sample using hybridization analysis, which is based on the specificity of nucleotide interactions. Oligonucleotides or cDNA can be used to selectively identify or capture DNA or RNA of specific sequence composition, and the amount of RNA or cDNA hybridized to a known capture sequence determined qualitatively or quantitatively, to provide information about the relative representation of a particular message within the pool of cellular messages in a sample. Hybridization analysis can be designed to allow for concurrent screening of the relative expression of hundreds to thousands of genes by using, for example, array-based technologies having high density formats, including filters, microscope slides, or microchips, or solution-based technologies that use spectroscopic analysis (e.g., mass spectrometry). One exemplary use of arrays in the diagnostic methods of the invention is described below in more detail.

Hybridization to arrays may be performed, where the arrays can be produced according to any suitable methods known in the art. For example, methods of producing large arrays of oligonucleotides are described in U.S. Pat. No. 5,134,854, and U.S. Pat. No. 5,445,934 using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in PCT published application no. WO 95/35505.

Methods for collection of data from hybridization of samples with an arrays are also well known in the art. For example, the polynucleotides of the cell samples can be generated using a detectable fluorescent label, and hybridization of the polynucleotides in the samples detected by scanning the microarrays for the presence of the detectable label. Methods and devices for detecting fluorescently marked targets on devices are known in the art. Generally, such detection devices include a microscope and light source for directing light at a substrate. A photon counter detects fluorescence from the substrate, while an x-y translation stage varies the location of the substrate. A confocal detection device that can be used in the subject methods is described in U.S. Pat. No. 5,631,734. A scanning laser microscope is described in Shalon et al., Genome Res. (1996) 6:639. A scan, using the appropriate excitation line, is performed for each fluorophore used. The digital images generated from the scan are then combined for subsequent analysis. For any particular array element, the ratio of the fluorescent signal from one sample is compared to the fluorescent signal from another sample, and the relative signal intensity determined.

Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e. data deviating from a predetermined statistical distribution, and calculating the relative binding affinity of the targets from the remaining data. The resulting data can be displayed as an image with the intensity in each region varying according to the binding affinity between targets and probes.

Pattern matching can be performed manually, or can be performed using a computer program. Methods for preparation of substrate matrices (e.g., arrays), design of oligonucleotides for use with such matrices, labeling of probes, hybridization conditions, scanning of hybridized matrices, and analysis of patterns generated, including comparison analysis, are described in, for example, U.S. Pat. No. 5,800,992.

In another screening method, the test sample is assayed for the level of a polypeptide. Diagnosis can be accomplished using any of a number of methods to determine the absence or presence or altered amounts of a differentially expressed polypeptide in the test sample. For example, detection can utilize staining of cells or histological sections (e.g., from a biopsy sample) with labeled antibodies, performed in accordance with conventional methods. Cells can be permeabilized to stain cytoplasmic molecules. In general, antibodies that specifically bind a differentially expressed polypeptide of the invention are added to a sample, and incubated for a period of time sufficient to allow binding to the epitope, usually at least about 10 minutes. The antibody can be detectably labeled for direct detection (e.g., using radioisotopes, enzymes, fluorescers, chemiluminescers, and the like), or can be used in conjunction with a second stage antibody or reagent to detect binding (e.g., biotin with horseradish peroxidase-conjugated avidin, a secondary antibody conjugated to a fluorescent compound, e.g. fluorescein, rhodamine, Texas red, etc.) The absence or presence of antibody binding can be determined by various methods, including flow cytometry of dissociated cells, microscopy, radiography, scintillation counting, etc. Any suitable alternative methods for qualitative or quantitative detection of levels or amounts of differentially expressed polypeptide can be used, for example ELISA, western blot, immunoprecipitation, radioimmunoassay, etc.

Kits may be provided, where the kit will comprise staining reagents that are sufficient to differentially identify the MPP cells described herein. A combination of interest may include one or more reagents specific for a marker or combination of markers of the present invention, and may further include antibodies specific for a lineage panel, CD34, CD38, CD90and CD45RA. The staining reagents are preferably antibodies, and may be detectably labeled. Kits may also include tubes, buffers, etc., and instructions for use.

Each publication cited in this specification is hereby incorporated by reference in its entirety for all purposes.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

### Experimental

### EXAMPLE 1

### Identification of a Hierarchy of Multipotent Hematopoietic Progenitors in Human Cord Blood

Mouse hematopoiesis is initiated by long-term hematopoietic stem cells (HSC) that differentiate into a series of multipotent progenitors that exhibit progressively diminished self-renewal ability. In human hematopoiesis, populations enriched for HSC activity have been identified, as have downstream lineage-committed progenitors, but multipotent progenitor activity has not been uniquely isolated. Previous reports indicate that human HSC are enriched in Lin−CD34+CD38− cord blood and bone marrow and express CD90. We demonstrate that the Lin−CD34+CD38− fraction of cord blood and bone marrow can be subdivided into three subpopulations: CD90+CD45RA−, CD90−CD45RA−, and CD90−CD45RA+. Utilizing in vivo transplantation studies and complementary in vitro assays, we demonstrate that the Lin−CD34+CD38−CD90+CD45RA− cord blood fraction contains HSC, and isolate this activity to as few as 10 purified cells. Furthermore, we report the first prospective isolation of a population of candidate human multipotent progenitors, Lin−CD34+CD38−CD90−CD45RA− cord blood.

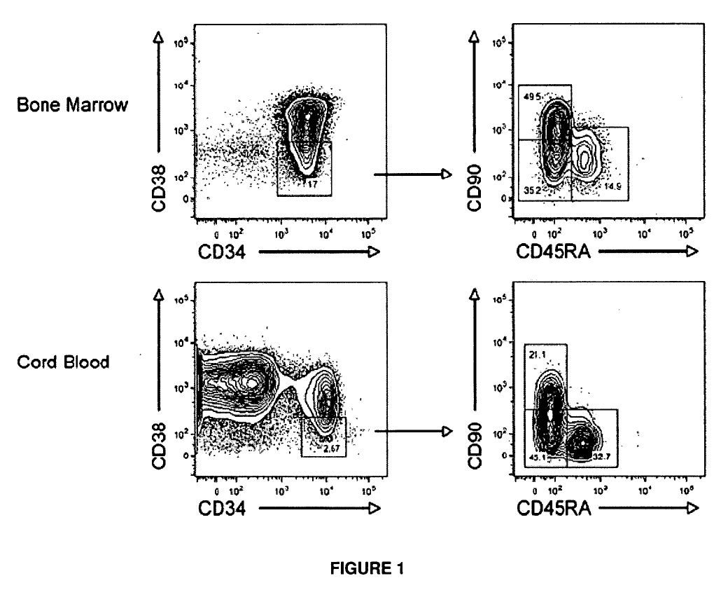

Identification of CD90/CD45RA Subpopulations of Lin−CD34+CD38− Human Bone Marrow and Cord Blood. Data from multiple investigators indicate that human HSC activity resides in the CD90+ fraction of Lin−CD34+CD38−/lo cells. Using the marker CD45RA, three subpopulations of Lin−CD34+CD38− bone marrow and cord blood were identified: (1) CD90+CD45RA−, (2) CD90−CD45RA−, and (3) CD90−CD45RA+ (FIG. 1). In the bone marrow these population comprised 30.3±18.9% (CD90+CD45RA−), 37.7±14.1% (CD90−CD45RA−), and 24.7±11.8% (CD90−CD45RA+) of Lin−CD34+CD38− cells (n=10). In cord blood, these fractions constituted 25.2±10.3% (CD90+CD45RA−), 49.8±11.4% (CD90−CD45RA−), and 18.4'8.4% (CD90−CD45RA+) of Lin−CD34+CD38− cells (n=22). All three subpopulations were isolated to >95% purity from cord blood and bone marrow by FACS.

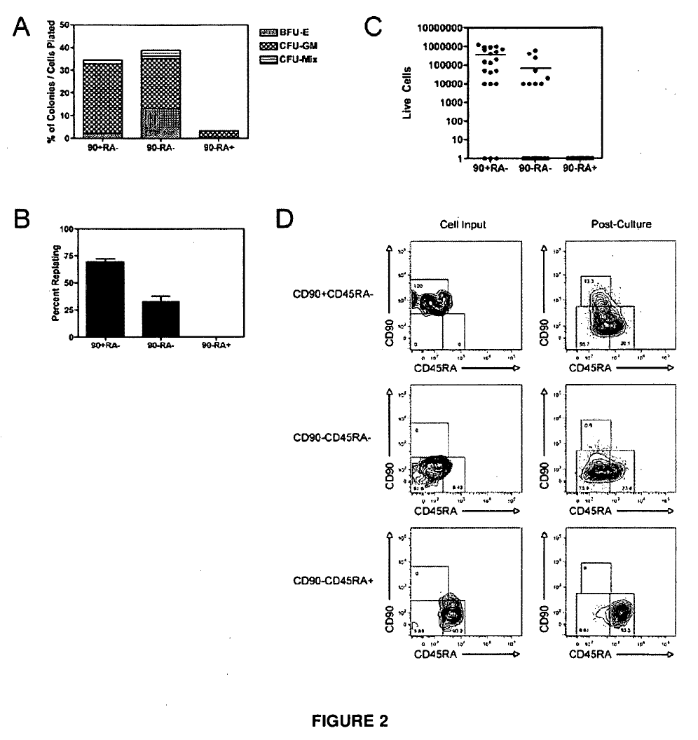

Methylcellulose Colony Formation and Replating of CD90/CD45RA Subpopulations. In order to assess the lineage potential of the CD90/CD45RA subpopulations, each was assayed for in vitro colony formation in methylcellulose. Single cells of each population were sorted into individual wells of a 96-well plate containing complete methylcellulose. In all cases, only a single colony was detected. CD90+CD45RA− cells formed all types of myeloid colonies, as did CD90−CD45RA− cells (FIG. 2A). No differences were detected in plating efficiency or colony subtype distribution between these two subpopulations; however, the CD90+CD45RA− colonies were generally much larger and faster growing. CD90−CD45RA+ cells formed very few colonies, suggesting that these cells possess limited myeloid differentiation potential.

All colonies derived from individual cells were then harvested, dissociated, and plated in complete methylcellulose in order to determine replating efficiency, an in vitro surrogate for self-renewal (FIG. 2B). 70% of colonies derived from CD90+CD45RA− cells were able to form new colonies (in most cases, hundreds) upon replating. 33% of colonies derived from CD90−CD45RA− cells were able to form new colonies (in most cases, fewer than 50). None of the colonies derived from CD90+CD45RA− cells were able to form new colonies; however, very few colonies formed in the first plating (n=6). Thus, the CD90−CD45RA− subpopulation is able to form all types of myeloid cells, but has reduced capacity for self-renewal compared to the CD90+CD45RA− subpopulation, which is presumed to contain HSC.

In Vitro Proliferation and Differentiation Identifies a Hierarchy Among the CD90/CD45RA Subpopulations. The CD90/CD45RA subpopulations were also assayed for in vitro proliferation in serum-free liquid culture. Single cells of each population were sorted into individual wells of a 96-well plate containing serum-free media supplemented with Flt-3 ligand, SCF, TPO, IL-3, and IL-6 and cultured for 2 weeks. At the end of the culture period, live cells were counted. CD90+CD45RA− cells proliferated extensively with a mean recovery of 345,000 cells; CD90−CD45RA− cells proliferated to a lesser degree with a mean recovery of 67,500 cells; CD90−CD45RA+ cells proliferated poorly with few live cells recovered (FIG. 2C). These results support the observed differences in methylcellulose colony size and suggest that the CD90− subpopulations are less primitive than the CD90+ subpopulation presumed to contain HSC.

In order to examine the potential hierarchical relationships between the CD90/CD45RA subpopulations within the Lin−CD34+CD38− fraction, each population was sorted in bulk into serum-free media supplemented with cytokines as described above. After four days in culture, the cells were re-analyzed for expression of CD90 and CD45RA (FIG. 2D). While CD90+CD45RA− cells gave rise to all three subpopulations, CD90−CD45RA− cells gave rise to both CD90− subpopulations, but not CD90+ cells. CD90−CD45RA+ cells gave rise principally to itself only. Together, these data establish an in vitro differentiation hierarchy in which CD90+CD45RA− cells give rise to CD90−CD45RA− cells, which in turn give rise to CD90−CD45RA+ cells.

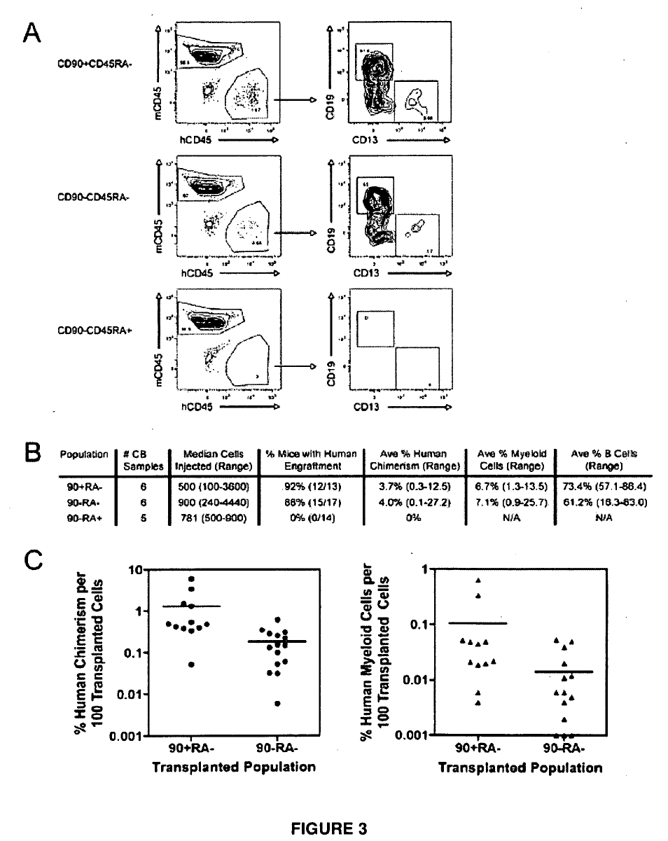

Long-Term In Vivo Multipotent Human Hematopoiesis with Transplantation of as few as 10 Lin−CD34+CD38−CD90+CD45RA− Cord Blood Cells. Newborn NOD/SCID/IL-2R^{γ}-null (NOG) mice were used in xenotransplantation assays to determine the differentiation potential and self-renewal ability of the CD90/CD45RA subpopulations. Transplantation of 100 purified CD90+CD45RA− cells resulted in circulating human CD45+ hematopoietic cells at 12 weeks, including both CD13+ myeloid cells, and CD19+ B cells, but not CD3+ T cells (FIG. 3A). Several of these mice were followed beyond 12 weeks (maximum 30 weeks), and all continued to have detectable human myeloid cells at similar levels in the peripheral blood (data not shown), indicating continued human engraftment. In order to isolate this activity to as few cells as possible, 10 purified CD90+CD45RA− cells were transplanted. At 12 weeks, few circulating human CD45+ cells were detected, and no CD13+ myeloid cells were present (FIG. 3A); however, analysis of the bone marrow showed significant human engraftment with both myeloid and lymphoid cells (FIG. 3A). Successful long-term human engraftment was observed in 3 out of 10 mice transplanted with 10 CD90+CD45RA− cells, with the 3 successful engraftments coming from independent cord blood samples.

Both CD13+ myeloid cells and CD19+ B cells were detected in the blood and bone marrow of engrafted mice, indicating that CD90+CD45RA− cells possess lymphoid and myeloid potential, and are likely multipotent. Analysis of the spleen from engrafted mice identified human CD45+CD3+ T cells, and staining of the bone marrow with glycophorin-A and CD61/41 identified human erythroid cells and platelets. To confirm this flow cytometry-derived lineage analysis, human CD45+ cells from both peripheral blood and bone marrow were FACS-purified and cytospin preparations were stained with Wright-Giemsa. These stains confirmed the presence of mature human lymphocytes, neutrophils, and monocytes in the peripheral blood (FIG. 3B, panels 1-3). In the bone marrow, both lymphocytes and maturing myeloid cells were readily detected (FIG. 3B, panel 4). Collectively, these data indicate that Lin−CD34+CD38−CD90+CD45RA− cord blood cells are capable of establishing long-term in vivo multipotent human hematopoiesis and that this activity can be isolated to as few as 10 cells.

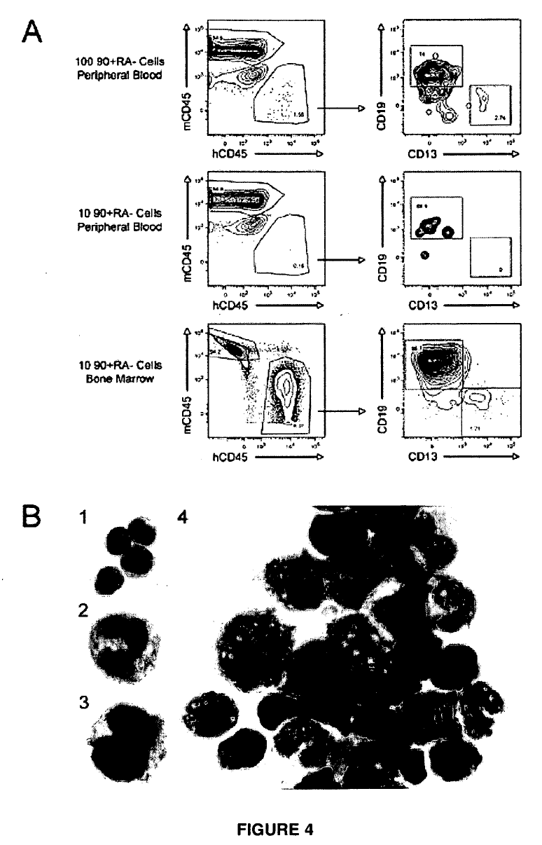

Long-Term In Vivo Multipotent Human Hematopoiesis Requires Transplantation of More CD90−CD45RA− Cells Than CD90+CD45RA− Cells. All three CD90/CD45RA subpopulations of cord blood were purified by FACS and transplanted into NOG mice. Transplantation of both the CD90+CD45RA− and the CD90−CD45RA− subpopulations resulted in detectable human myeloid and B lymphoid cells in the peripheral blood 12 weeks after transplantation (FIG. 4A). No human cells were detected in the peripheral blood of mice transplanted with the CD90−CD45RA+ subpopulation, even at time points as early as 4 weeks after transplantation (FIG. 4A). Multiple transplantation experiments were conducted with independent cord blood samples resulting in transplantation of between 100 and 4440 cells of each population (FIG. 4B). The cumulative data from these experiments showed that 12 of 13 mice (92%) transplanted with CD90+CD45RA− cells, and 15 of 17 mice (88%) transplanted with CD90−CD45RA− cells contained human CD45+ cells in the peripheral blood at least 12 weeks after transplantation (FIG. 4B). In the engrafted mice, the average human CD45+ chimerism was 3.7% for the CD90+CD45RA− transplants compared to 4.0% for the CD90−CD45RA− transplants; the percentage of myeloid cells among human CD45+ cells was 6.7% for the CD90+CD45RA− transplants compared to 7.1% for the CD90−CD45RA− transplants (FIG. 4B). Because different cell numbers were used in each of these transplantation experiments, the engraftment per 100 transplanted cells was determined. The CD90+CD45RA− engrafted mice averaged 7 fold greater human chimerism and 7 fold greater human myeloid cells than the CD90−CD45RA− engrafted mice (FIG. 4C). The difference in human chimerism was statistically significant with p=0.02, while the difference in human myeloid cells approached statistical significance with p=0.08.

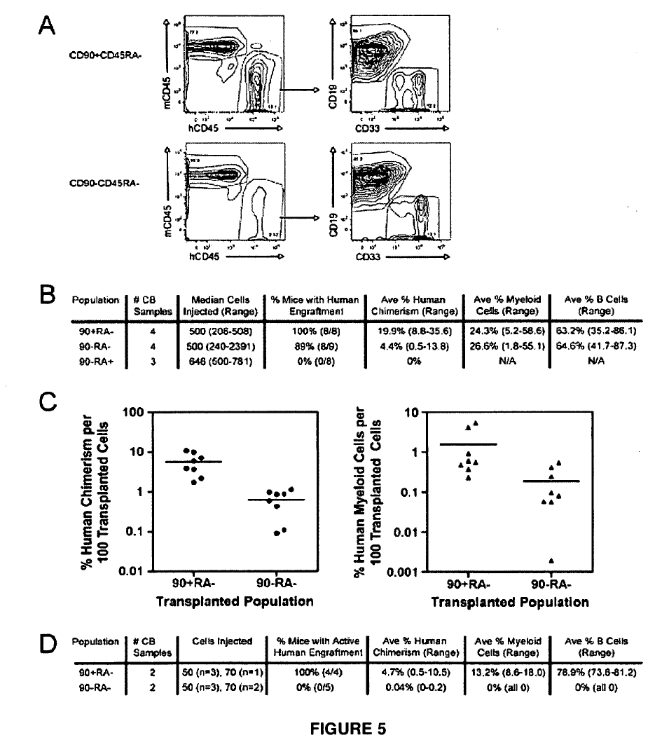

Analysis of the bone marrow of transplanted mice revealed human myeloid and B cells 12 weeks after transplantation of CD90+CD45RA− and CD90−CD45RA− cells (FIG. 5A). No human cells were detected in the bone marrow of mice transplanted with the CD90−CD45RA+ population. Cumulative data showed that 8 of 8 mice (100%) transplanted with CD90+CD45RA− cells, and 8 of 9 mice (89%) transplanted with CD90−CD45RA− cells contained human CD45+ cells in the bone marrow at least 12 weeks after transplantation (FIG. 5B). In the engrafted mice, the average human chimerism was 19.9% for the CD90+CD45RA− transplants compared to 4.4% for the CD90−CD45RA− transplants; the percent myeloid of total human CD45+ cells was 24.3% for the CD90+CD45RA− transplants compared to 26.6% for the CD90−CD45RA− transplants (FIG. 5B). In order to normalize for cell number, bone marrow engraftment per 100 transplanted cells was determined. The CD90+CD45RA− engrafted mice averaged 9 fold greater human chimerism and 9 fold greater human myeloid cells than the CD90−CD45RA− engrafted mice (FIG. 5C). The difference in human chimerism was statistically significant with p=0.001, while the difference in human myeloid cells approached statistical significance with p=0.07.

Analysis of the spleens of CD90+CD45RA− and CD90−CD45RA− transplanted mice identified human CD45+ cells consisting of rare myeloid cells, numerous B cells, and occasional T cells. No human cells were detected in the spleens of mice transplanted with CD90−CD45RA+ cells. Significant numbers of T cells were detected in only a subset of mice transplanted with either engrafting population (Supplementary FIG. 2). Determining splenic engraftment per 100 transplanted cells revealed that CD90+CD45RA− engrafted mice averaged 7 fold greater human chimerism than the CD90−CD45RA− engrafted mice, and this difference was statistically significant; however, no statistically significant difference was detected in the T cell percentage. Finally, bone marrow from mice engrafted with CD90−CD45RA− cells was found to contain GPA-positive human erythroid cells and CD61/CD41-positive human platelets (data not shown), indicating that these cells are multipotent.

To investigate the minimum number of cells required for successful engraftment, 50 or 70 CD90−CD45RA− or CD90+CD45RA− cells were double FACS-purified from 2 independent cord blood samples and transplanted into NOG mice. At least 11 weeks later, bone marrow was analyzed for human engraftment. All mice (n=4) transplanted with CD90+CD45RA− cells contained human myeloid and B cells, while no mice (n=5) transplanted with CD90−CD45RA− cells did (FIG. 5D). This difference was statistically significant with p=0.008. One of the CD90−CD45RA− transplanted mice did contain a small population (0.2%) of human T cells, indicating that it must have engrafted at an early time point. To directly investigate early engraftment, 50 double FACS-purified cells were transplanted into NOG newborn mice. 4 weeks later, bone marrow of all transplanted mice, both CD90+CD45RA− (n=2) and CD90−CD45RA− (n=2), contained human myeloid and B cells.

In summary, both the CD90+CD45RA− and CD90−CD45RA− subpopulations are capable of establishing long-term in vivo multipotent human hematopoiesis, with similar percentages of myeloid and lymphoid cell production. However, the CD90−CD45RA− cells have a lower engraftment capacity as they require transplantation of more cells for long-term engraftment and generate fewer human cells per cell-equivalent transplant.

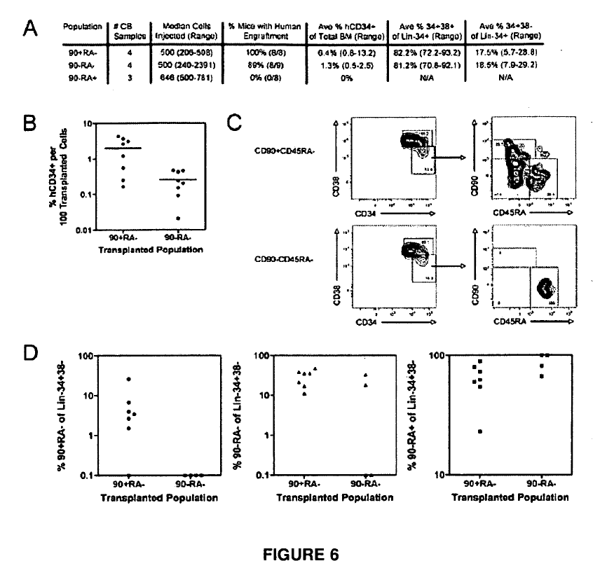

In Vivo Analysis of Human CD34+ Cells Identifies a Hierarchy Among the CD90/CD45RA Subpopulations. In order to assess the hierarchical relationships among the CD90/CD45RA subpopulations in vivo, human CD34+ progenitor cells from the bone marrows of engrafted mice were analyzed 12 weeks after transplantation. In mice transplanted with CD90+CD45RA− cells the bone marrow contained, on average, 6.4% human CD34+ cells compared to 1.3% human CD34+ cells in mice transplanted with CD90−CD45RA− cells (FIG. 6A). CD34+ engraftment per 100 transplanted cells averaged 8 fold greater in CD90+CD45RA− engrafted mice than CD90−CD45RA− engrafted mice, and this difference was statistically significant (FIG. 6B). This 8 fold statistically significant difference was also present when comparing the percentage of Lin−CD34+ cells in total bone marrow (data not shown). Within the engrafted Lin−CD34+ fraction, there were no differences between CD90+CD45RA− and CD90−CD45RA− engrafted mice with respect to the percentages of CD34+CD38+or CD34+CD38− cells (FIG. 6A). Thus, CD90−CD45RA− cells appear to be able to generate progenitor cells in similar proportions to CD90+CD45RA− cells, but are less efficient in doing so in vivo.

Human Lin−CD34+CD38− cells in the bone marrow of engrafted mice were also analyzed for the expression of CD90 and CD45RA. All three CD90/CD45RA subpopulations were detected in the bone marrow of mice transplanted with CD90+CD45RA− cells; however, only the two CD90− subpopulations were detected in mice transplanted with CD90−CD45RA− cells (FIG. 6C, D). In some mice transplanted with CD90−CD45RA− cells, only CD90−CD45RA+ cells were detected. Together, these data establish an in vivo differentiation hierarchy among Lin−CD34+CD38− cord blood cells proceeding from CD90+CD45RA− to CD90−CD45RA− to CD90−CD45RA+ cells.

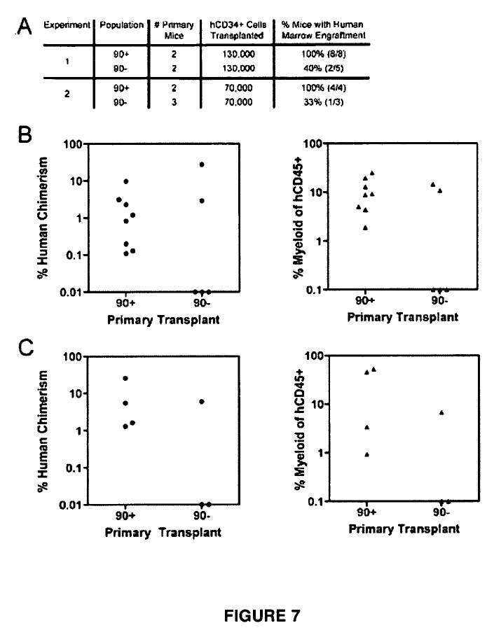

Enrichment of Secondary Transplant Ability in the CD90+CD45RA− Subpopulation. Self-renewal is the key property distinguishing HSC from MPP, and is defined by the ability to sustain long-term hematopoiesis and generate successful secondary transplants. Since both the CD90+CD45RA− and CD90−CD45RA− subpopulations demonstrated the ability to establish long-term in vivo multipotent hematopoiesis, we next assessed their ability to generate successful secondary transplants. Human CD34+ cells were purified from whole bone marrow of primary engrafted mice and equal numbers of CD34+ cells were transplanted into NOG newborn mice. Bone marrows from these secondary recipients were analyzed at least 10 weeks after transplantation. In one experiment, 130,000 human CD34+ cells were transplanted into secondary recipients. Human CD45+ cells and human myeloid cells were detected in 8 of 8 mice (100%) from CD90+ primary transplants, but only 2 of 5 mice (40%) from CD90− primary transplants (FIG. 7A,B). In a second experiment, 70,000 human CD34+ cells were transplanted into secondary recipients. Human CD45+ cells and human myeloid cells were detected in 4 of 4 mice (100%) from CD90+ primary transplants, but only 1 of 3 mice (40%) from CD90− primary transplants (FIG. 7A,C). This difference, 12 of 12 (100%) for CD90+ versus 3 of 8 (37.5%) for CD90−, was statistically significant with p=0.004. These data indicate that the CD90+CD45RA− subpopulation is enriched for the ability to generate successful secondary transplants compared to the CD90−CD45RA− subpopulation.

HSC are defined by two key functional properties: (1) multipotency, defined as the ability to form all differentiated blood cells, and (2) long-term self-renewal, defined as the ability to give rise to progeny identical to the parent through cell division. It is this property of self-renewal that distinguishes HSC from multipotent progenitors, and is experimentally demonstrated through the ability to generate successful secondary transplants. We show here that both CD90+CD45RA− and CD90−CD45RA− cord blood cells are able to establish long-term multipotent hematopoiesis in vivo, and that the CD90+CD45RA− subpopulation is enriched for the ability to generate successful secondary transplants. We conclude that the CD90+CD45RA− subpopulation contains HSC, while the CD90−CD45RA− subpopulation contains multipotent progenitors. This represents the first identification and prospective isolation of a population of human multipotent progenitors.

Both the CD90+CD45RA− and CD90−CD45RA− cells are able to establish multipotent long-term human hematopoiesis in vivo; however, the CD90−CD45RA− subpopulation requires more cells to accomplish this as indicated by the failure of 50 and 70 cell transplants to long-term engraft, unlike the CD90+CD45RA− subpopulation which engrafts long-term with as few as 10 cells. The fact that 50 CD90−CD45RA− cells show myeloid and B lymphoid engraftment at 4 weeks, but not 12 weeks, indicates that this fraction contains non-HSC multipotent cells. Thus, we have demonstrated that CD90−CD45RA− cells are multipotent, exhibit a reduced and incomplete capacity for self-renewal, and lie downstream of CD90+CD45RA− cells in the hematopoietic hierarchy. We conclude that CD90−CD45RA− cells are a multipotent hematopoietic progenitor.

Hematopoiesis proceeds through an organized hierarchy in which lineage potential becomes increasingly restricted and a given population can only give rise to downstream populations. We investigated the hierarchical relationships between the CD90/CD45RA subpopulations of Lin−CD34+CD38− cord blood and found that both in vitro and in vivo, the CD90+CD45RA− population gives rise to itself and both the CD90−CD45RA− and CD90− CD45RA+ subpopulations. CD90−CD45RA− cells do not give rise to CD90+ cells, but can form both CD90− subpopulations. In vitro the CD90−CD45RA+ cells give rise principally to itself only. These results establish a hierarchy among Lin−CD34+CD38− cord blood cells in which CD90+CD45RA− cells are upstream of CD90−CD45RA− cells, which are upstream of CD90−CD45RA+ cells.

CD90 and CD45RA identify a third population within the Lin−CD34+CD38− fraction of cord blood and bone marrow, the CD90−CD45RA+ subpopulation. Transplantation with up to 900 of these cells does not result in any circulating human hematopoietic cells at 4 weeks after transplantation, and there are no detectable human cells in the bone marrow at 12 weeks. Furthermore, these cells have extremely poor methylcellulose colony forming ability, yielding only 6 colonies from 180 plated cells, suggesting that they possess limited myeloid differentiation potential. These cells also did not proliferate in liquid culture under conditions able to promote the growth of the other two subpopulations. It is interesting to note, that cells with this immunophenotype can be found within the bone marrow of mice engrafted with either the CD90+CD45RA− or CD90−CD45RA− subpopulation. Thus, it is likely that they contribute to ongoing human hematopoiesis, but at this time cannot be placed within the hematopoietic hierarchy.

Numerous xenotransplantation experiments have reported the successful enrichment of human HSC activity in Lin−CD34+CD38−/lo fractions of human hematopoietic progenitors through the demonstration of long-term multipotent engraftment and successful secondary transplantation. In published reports, successful secondary engraftment has required primary transplantation of large numbers of cells, minimally thousands and usually many more. We report here long-term in vivo human engraftment and successful secondary engraftment with transplantation of 500 purified Lin−CD34+CD38−CD90+CD45RA− cord blood cells. The major differences between our results and previous reports are: (1) the use of the NOG newborn mice, which appear to be well-suited for human hematopoietic engraftment, and (2) the combination of Lin−CD34+CD38−CD90+CD45RA− markers for HSC purification. With this immunophenotype and xenotransplantation assay, we have directly isolated cord blood HSC activity to fewer cells than in previous reports.

Implications for Human Acute Myeloid Leukemia. Analogous to normal hematopoiesis, human acute myeloid leukemia (AML) is organized as a hierarchy initiated by leukemia stem cells (LSC) that are able to self-renew and give rise to all the cells within the leukemia (Tan et al. (2006) Lab Invest 86, 1203-1207; Wang and Dick (2005) Trends Cell Biol 15, 494-501). In a series of xenotransplantation experiments, Dick and colleagues first demonstrated the existence of human AML LSC and localized them to the Lin−CD34+CD38− fraction of AML (Bonnet and Dick (1997) Nat Med 3, 730-737; Lapidot et al. (1994) Nature 367, 645-648). Based on these observations, a model was proposed in which HSC are the cell of origin for AML LSC. However, subsequent experiments indicated that AML LSC, unlike HSC, do not express CD90 (Miyamoto et al. (2000) Proc Natl Acad Sci U S A 97, 7521-7526). There are two hypotheses to account for this difference: (1) AML LSC are indeed derived from HSC, but have aberrantly lost expression of CD90, or (2) AML LSC do not derive from HSC but instead come from a downstream progenitor that lacks expression of CD90.

Evidence supporting the second hypothesis comes from previous studies of AML1-ETO translocation-associated AML in atom bomb survivors from Hiroshima. The AML LSC were contained in the Lin−CD34+CD38−CD90− fraction (Miyamoto et al. (2000) PNAS 97:7521). However, when the bone marrow of long-term disease-free survivors was examined, the AML1-ETO translocation was detected in Lin−CD34+CD38−CD90+ non-leukemic HSC. This demonstrates that pre-leukemic genetic changes can take place within HSC, but ultimate transformation to AML LSC requires additional mutations that do not occur in HSC, but instead take place in a CD90− downstream population.

Why does this matter? If the normal counterpart to long-term self-renewing AML LSC is not capable of long-term self-renewal itself, then AML LSC must have undergone mutational or epigenetic activation of a self-renewal pathway. These changes, when identified, become targets for therapeutic intervention to eradicate the LSC. Evidence for aberrant activation of self-renewal in LSC comes from studies of human blast crisis CML, where normally non-self-renewing cells transform into LSC in part through activation of the Wnt/beta-catenin pathway (Jamieson et al. (2004) N Engl J Med 351, 657-667). Through comparisons between AML LSC and the newly identified MPP, genetic and/or epigenetic events that lead to the transformation of MPP into AML LSC can be identified.

### Experimental Procedures

Human Samples. Normal human bone marrow mononuclear cells were purchased from AllCells Inc. (Emeryville, Calif.). Human cord blood was collected from placentas and/or umbilical cords obtained from the Stanford Medical Center, according to an IRB-approved protocol (Stanford IRB# 4637). Mononuclear cells were prepared using Ficoll-Paque Plus (GE Healthcare, Fairfield, Conn.), and cryopreserved in 90% FBS/10% DMSO. All experiments were conducted with cryopreserved cord blood cells that were thawed and washed with IMDM containing 10% FBS. In some cases, CD34+ cells were enriched using MACS (Miltenyi Biotec, Germany) or Robosep (Stem Cell Technologies, Canada) immunomagnetic beads.

Flow Cytometry Analysis and Cell Sorting. A panel of antibodies was used for analysis and sorting of progenitor subpopulations, as well as lineage analysis of human chimerism/engraftment, and used to stain cell suspensions (Supplementary Methods). Cells were either analyzed or sorted using a FACS Aria cytometer (BD Biosciences). Analysis of flow cytometry data was performed using FlowJo Software (Treestar, Ashland, Oreg.). Statistical analysis using Student's t-test or Fisher's exact test was performed with Microsoft Excel and/or GraphPad Prism (San Diego, Calif.) software.

In Vitro Assays: Cytology, Methylcellulose, and Liquid Culture. For cytologic analysis, sorted cells were centrifuged onto slides using a Shandon Cytocentrifuge 4 (Thermo Scientific, Waltham, Mass.), and stained with Wright-Giemsa. Photomicrographs were taken using a 100× objective under oil.

Methylcellulose colony formation was assayed by clone-sorting single cells into individual wells of a 96-well plate, each containing 100 μl of complete methylcellulose (Methocult GF+ H4435, Stem Cell Technologies). Plates were incubated for 12-14 days at 37° C., then scored based on morphology. All colonies were harvested, dissociated by resuspending in sterile PBS, and replated into individual wells of a 24-well plate, each containing 500 μl of complete methylcellulose. Plates were incubated for 12-14 days at 37° C., after which replating was determined by assessing colony formation. Statistical analysis using Student's t-test was performed with Microsoft Excel and/or GraphPad Prism (San Diego, Calif.) software.

In vitro proliferation was assayed by clone-sorting single cells into individual wells of a 96-well plate, each containing 100 μl of StemSpan media (Stem Cell Technologies), supplemented with 40 μg/ml human LDL (Sigma-Aldrich) and cytokines (R&D Systems, Minneapolis, Minn.): 100 ng/ml Flt-3 ligand, 100 ng/ml SCF, 50 ng/ml TPO, 20 ng/ml IL-3, and 20 ng/ml IL-6. Plates were incubated for 14 days at 37° C., after which live cells were counted by trypan blue exclusion. For in vitro differentiation assays, cells were sorted in bulk into this same culture media and incubated for 3-4 days at 37° C., after which cells were harvested and analyzed by flow cytometry. Statistical analysis using Student's t-test was performed with Microsoft Excel and/or GraphPad Prism (San Diego, Calif.) software.

Mouse Transplantation. NOD.Cg-Prkdc^{scid}II2rg^{tm1Wjl}/SzJ mice (NOG) were obtained from The Jackson Laboratory (Bar Harbor, Me.) and bred in a Specific Pathogen-Free environment per Stanford Administrative Panel on Laboratory Animal Care guidelines (Protocol 10725). P0-P2 newborn pups were conditioned with 100 rads of gamma irradiation up to 24 hours prior to transplantation. Desired cells were resuspended in 20-40 ul of PBS containing 2% FBS and transplanted intravenously via the anterior facial vein using a 30 or 31 gauge needle. For secondary transplants, human CD34+ bone marrow cells from primary engrafted mice were enriched using MACS immunomagnetic beads (Miltenyi Biotec), and transplanted into newborn NOG recipients.

Antibodies and Staining of Cell Suspensions. A panel of antibodies was used for analysis and sorting of progenitor populations including PE-Cy5 conjugated anti-human lineage markers: CD3, S4.1; CD4, S3.5; CD7, CD7-6B7; CD8, 3B5; CD10, 5-1B4; CD14, TUK4; CD19, SJ25-C1, CD20, 13.6E12 (Caltag/Invitrogen, Carlsbad, Calif.); CD2, RPA-2.10; CD11b, ICRF44; CD56, B159; GPA, GA-R2 (BD Biosciences, San Jose, Calif.), as well as PB-conjugated anti-CD45RA, MEM56 (Caltag); FITC anti-CD38, HIT2 (Caltag); PE-conjugated anti-CD90(Thy-1), 5E10 (BD Biosciences); APC-conjugated anti-CD34, 8G12 (BD Biosciences).

Lineage analysis of human chimerism/engraftment was performed using anti-human antibodies: PB-conjugated CD45, H130; APC-Alexa Fluor 750-conjugated CD3, S4.1; APC-conjugated CD19, SJ25-C1; PE-conjugated CD13, TK1 (Caltag); PE-conjugated CD33, P67.6, PE-conjugated GPA, GA-R2, APC-conjugated CD41a, HIP8 (BD Biosciences). Mouse leukocytes and red cells were identified based on the expression of Alexa488 or PE-Cy7-conjugated CD45.1, clone A20.1.7, and PE-Cy5 or PE-Cy7-conjugated Ter119 (eBiosciences, San Diego, Calif.), respectively.

Single-cell suspensions were prepared by standard methods from the peripheral blood, spleen, and bone marrow of transplanted mice. All preparations underwent hypotonic red cell lysis using ACK; bone marrow mononuclear cells were isolated using Histopaque-1119 (Sigma-Aldrich, St. Louis, Mo.). For both analysis and sorting, cells were stained with the desired antibody combinations on ice for 30 minutes, and dead cells were excluded by propidium iodide staining.

What is claimed is:

1. A substantially pure composition of human multipotent progenitor cells, wherein at least 80% of the cells in said composition are characterized as CD34+CD38−CD90−CD45RA− and lacking the expression of lineage specific markers.

2. The composition according to claim 1, wherein said lineage specific markers include CD2, CD3; CD4; CD7; CD8; CD10; CD11b; CD14; CD19; CD20; CD56; and glycophorin A (GPA).

3. The composition of claim 1, wherein said multipotent progenitor cells, when cultured in complete methylcellulose give rise to all types of myeloid cells.

4. The composition of claim 1, wherein said multipotent progenitor cells give rise to differentiated hematopoietic cells for at least about 4 weeks.

5. The composition of claim 1, wherein said cells are genetically modified to comprise an exogenous DNA vector.

6. A method of enrichment for a composition of human multipotent progenitor cells, wherein at least 80% of the cells in said composition are characterized as CD34+CD38−CD90−CD45RA− and lacking the expression of lineage specific markers, the method comprising:
combining reagents that specifically recognize CD34, CD38, CD90, CD45RA and lineage specific markers with a sample of hematopoietic cells; and
selecting for those cells that are CD34+CD38−CD90−CD45RA− and lacking the expression of lineage specific markers to provide an enriched population of cells having multipotent progenitor activity.

7. The method according to claim 6, wherein said sample of hematopoietic cells is human bone marrow.

8. The method according to claim 6, wherein said sample of hematopoietic cells is human cord blood.

9. The method according to claim 6, wherein said sample of hematopoietic cells is mobilized peripheral blood.

10. A method of screening for genetic sequences specifically expressed in cells committed to the multipotent lineage, the method comprising:
isolating RNA from a cell population according to claim 1,
generating a probe from said RNA,
screening a population of nucleic acids for hybridization to said probe.

11. A method of providing differentiated hematopoietic cells to a mammalian recipient, the method comprising:
administering to said recipient a population of multipotent progenitor cells, wherein at least 80% of the cells in said population are characterized as CD34+CD38−CD90−CD45RA− and lacking the expression of lineage specific markers,
wherein said multipotent progenitor cells give rise to differentiated hematopoietic cells in vivo.

12. A method of screening for factors that affect hematopoiesis, the method comprising:
combining a candidate hematopoiesis factor with a population of multipotent progenitor cells, wherein at least 80% of the cells in said population are characterized as CD34+CD38−CD90−CD45RA− and lacking the expression of lineage specific markers, and
determining the effect of said agent on the formation of myeloid and lymphoid cells.
